(19) United States
(12) Patent Application Publication
(10) Pub. No.: US 2002/0172688 A1
(43) Pub. Date: Nov. 21, 2002

(54) HLA-restricted cytotoxic T-Lymphocytevaccine for prevention of HIV infection

(76) Inventor: Bruce Lyday, Garden Grove, CA (US)

Correspondence Address: Bruce W. Lyday, 12851 Haster St. #1-D, Garden Grove, CA, 92840, US

(21) Appl. No.: 09/809,820

(22) Filed: Mar. 16, 2001

(51) Int. Cl. A61K 39/21; A61K 39/12; C12N 7/00

(52) U.S. Cl. 424/204.1; 424/208.1; 435/235.1

(57) ABSTRACT

An improved method of inducing protective immunity to the Human Immunodeficiency Virus through isolating a person's antigen-presenting cells from blood, then pulsing these cells with short peptides that bind the person's Class I Major Histocompatibility Complex Types and correspond to conserved segments of the HIV structural and functional genes. These pulsed dendritic cells are then injected intravenously, where they will travel to lymph tissue and prime HIV-specific Cytotoxic T Lymphocytes. The immune system is then activated by injecting the person with a weakened arbovirus, Chikungunya. The interferons and other immune-activating chemicals induced by the virus will stimulate creation of billions of killer cells that recognize HIV proteins, allowing for rapid mobilization of memory immune cells to contain and eliminate any subsequent infection with the HIV virus. The vaccine is custom-designed to each person and uses no HIV virus for safety purposes. It is understood that the examples an embodiments described herein are for illustrative purposes only and various changes or modifications in light thereof will be suggested to persons skilled in the art and are to be included within the spirit and purview of this application and scope of the appended claims.

### BACKGROUND—FIELD OF INVENTION

The invention relates to a cell-mediated vaccine to prevent infection with HIV, the Human Immunodeficiency Virus.

### BACKGROUND—DESCRIPTION OF PRIOR ART

Despite almost twenty years of intensive research and billions of dollars spent on developing a safe and effective vaccine for HIV, no such vaccine exists. The antiviral vaccines approved for human use are either killed or live, attenuated (weakened) viruses that provoke neutralizing antibodies so that the invading virus cannot infect cells. Because of the deadly nature of HIV, any vaccine containing live viruses is prima facie unsafe, and would never obtain FDA approval. A vaccine containing killed HIV is subject to the same safety concerns, as methods of virus inactivation (radiation, heat, formalin) are not 100% effective.

For these reasons, research efforts have focused on protein sub-unit vaccines. Using Recombinant DNA technology, scientists can reproduce the proteins in bacteria such as E. coli. These proteins, when injected into the human body, induce antibodies. Because zero HIV is present, these vaccines are considered very safe.

Unfortunately, sub-unit vaccines do not provoke a strong neutralizing antibody response. Also, they do not induce formation of memory Cytotoxic T-Lymphocyte Killer cells (mCTL). These killer cells use a specific T-Cell Receptor (TCR) to identify and destroy viral-infected cells. An ideal vaccine would result in both neutralizing antibody and memory CTL against the dominant viral proteins.

An additional complication to vaccine development is HIV's status as a retrovirus, a pathogen with a complex infection and replication cycle. Viruses are classified as DNA or RNA viruses depending upon their nucleic acid type. DNA viruses (Herpes, Cowpox,

Rabies), use host enzymes to transcribe their DNA to RNA, and then to protein. RNA Viruses (Influenza, Yellow Fever) bypass the transcription stage, and carry their own enzymes to facilitate converting their RNA to protein. Retroviruses carry RNA, but use a unique enzyme called Reverse Transcriptase to synthesize DNA from their RNA. A second enzyme, Integrase, inserts the DNA strand into the genome of the host cell. The DNA is transcribed and translated into viral proteins by host enzymes. These additional steps, with error rates ranging from 0.000001 to 0.001 per strand synthesis, contribute to a high mutation rate of HIV. There are now hundreds of serologically distinct strains of HIV, divided into seven clades, or virus families.

The most important antibody target of HIV is the main envelope spike, gp120, signifying a glycoprotein with a molecular weight of 120,000 daltons. This large protein has 20 separate sites where a branched sugar chain can be attached to an asparaginine amino acid residue. By adding or deleting a sugar chain, HIV can change its spike structure so that antibody will no longer bind to, and neutralize the virus. Experts in the field agree that a vaccine directed against the envelope spike gp120 is impossible.

The core proteins of HIV, p15, p17, and p24, are more stable than the envelope spike, and may be better vaccine targets. Unfortunately, because they are located within the virus, antibodies against core proteins do not neutralize the virus. Infected cells express these core proteins in amounts sufficient for CTL recognition and destruction, so a cell-mediated memory killer cell vaccine directed against the core proteins of HIV could satisfy requirements of safety and effectiveness.

The most stable of the HIV core proteins is the dominant antigen p24, signifying a non-glycosolated protein with a molecular weight of 24,000 daltons. The absence of asparaginine-linked sugar chains contributes to its genetic stability, but other factors place limits on its mutation frequency.

Consider a skyscraper, a building with both internal and external structural design characteristics, which can be further classified as critical, or non-critical. An example of a non-critical factor would be the choice of smoked-gray windows vs. mirrored windows, or the location of the company flagpoles on the roof. These can be changed without altering the basic structural integrity of the building. HIV virus is also free to alter the position of the sugar chains on gp120 without compromising the integrity of the structure.

Consider the substitution of aluminum or low-grade steel I-beams for the skyscraper frame. The building's structural integrity has now been compromised, and it will probably collapse. HIV is limited in its mutation capability for the critical core proteins because it is a physical entity whose structure, however small, is bound by the same physics constraints of the skyscraper. Mutations in this region will most likely result in a virion core that will be defective, and will not self-assemble into an intact virus. The main core protein p24 is highly conserved among HIV strains, and represents the Achilles Heel that can be exploited by creative vaccine developers.

### OBJECTS OF ADVANTAGES

Accordingly, several objects and advantages of the invention are:

(a) to provide a cell-mediated, memory Cytotoxic T Lymphocyte clone capable of recognizing the dominant antigenic determinants of p24 and other conserved proteins, killing any cells which express these determinants associated with the Major Histocompatibility Complex (MHC) proteins on the infected cell surface.

(b) to provide protection against all known strains of HIV by using peptides from the various HIV clades that efficiently bind the person's HLA (Human Leukocyte Antigen) gene products.

(c) to accomplish these goals safely by using peptide-pulsed Antigen Presenting Cells (APC) to prime the T cells, eliminating the need for any whole HIV Virus.

(d) to achieve maximum T-cell activation by following the infusion of the peptide-pulsed APC with an attenuated arbovirus, Chikungunya.

Further Objects and Advantages of the invention will become apparent from a consideration of the ensuing description.

### SUMMARY OF INVENTION

The invention describes a method for making and using a safe, effective vaccine for prevention of infection with the Human Immunodeficiency Virus. The invention uses modem technology in the fields of Virology and Immunology to induce a large population of MHC-restricted Cytotoxic T Lymphocytes with receptors for the antigenic determinants of the main HIV core protein p24. These antigens are matched to the person's HLA type, and pulsed into Antigen-Presenting Cells, which are infused into the person's veins. The person is then injected with a weakened strain of Chikungunya Virus , which activates the T-Cells through Interleukin-2. Activated T Cells then make contact with the pulsed APC, and become specific for the HIV epitopes. These memory CTL persist for life, and any subsequent HIV infection will be prevented by the pre-existing CTL population. The vaccine combines safety and effectiveness that conforms to FDA standards.

### DESCRIPTION OF INVENTION

Chikungunya Virus is an RNA Virus of the Togavirus Family, subfamily Alphavirus (Group A). It has an icosahederal geometry, is approximately 40-45 nanometers in diameter, and has two major envelope proteins. The E1 or Hemagglutinin protein is very rich in the hydrophobic amino acid lysine, and has a molecular weight of approximately 45,000 daltons. The E2 or Neurmainidase protein is rich in the amino acids alanine, serine, and valine, and has a molecular weight of approximately 50,000 daltons. The E3 protein is a transmembrane structure that anchors the E1 and E2 proteins to the virus core proteins. Neutralizing antibodies are primarily directed against the E1 protein.

Dendritic cells are derived from bone marrow or monocyte precursors, and are distinguished by their CD1 and CD34 protein markers, and numerous protoplasmic processes. They are devoid of Class I MHC markers, so allogenic cells (from different donors) may be safely used to prime a person's T Cells at the ratio of 1:1,000.

The peptides used in the invention are short (7-10) amino acid chains synthesized by laboratory methods to be identical to the T-Cell recognition sites on the HIV proteins. These peptides are comprised of hydrophobic, small side-chain amino acids that conform to the vaccinee's HLA gene products using a Peptide-Binding Assay.

### Examples of p24 Epitopes that Bind Human HLA Gene Products

| Peptide | HLA-Restriction | Inhibition of CTL by Antibody |
|---|---|---|
| 77-85 | HLA-A2 | Not Determined |
| 133-147 | HLA-A2, B27, B37 | 96% |
| 263-272 | HLA-A2, B27, B37 | 53% |
| 263-277 | HLA-A2, B27, B37 | 32% |
| 270-287 | HLA-A2, B27, B37 | 100% |
| 319-337 | HLA-A2, B27, B37 | 92% |
| 335-351 | HLA-A2, B37 | 33% |

### Operation of Invention—Theory of Operation

The invention circumvents barriers to an HIV vaccine by using allogenic anttigen-presenting Dendritic Cells (DC), loaded with the immunodominant peptides of HIV core protein p24, coupled with an attenuated arbovirus culture to activate the T Cell system. After matching the person's HLA gene products to the p24 peptides, these peptides are pulsed into the Dendritic Cells in vitro. These peptide-loaded DC are then infused into the person's veins, where they migrate to lymph nodes. Injection of the Chikungunya Virus activates the T cells through Interleukin-2, and proliferating T Cells make contact with the DC, and become specific for p24 epitopes. This results in a large clone of p24-specific killer cells, some of which will persist for life as memory CTL. Any subsequent exposure to HIV will active these mCTL, which will rapidly proliferate to destroy infected T4 lymphocytes before an infection gains a foothold. Because the CTL are programmed for p24, HIV cannot mutate these regions and still produce intact viruses capable of self-assembly and infection. Numerous articles in the Scientific Literature confirm that p24 is the most stable of HIV Gag proteins, and deletions or mutations in large sequences of p24 prevent assembly of infection-competent viruses.

Additional literature studies have confirmed that CTL specific for Gag p24 epitopes have provided protective immunity in babies born to HIV-infected mothers. These p24-specific CTL have demonstrated cross-clade lytic activity against HLA-matched cells infected with African Clade A, or North American Clade B viruses. This is critical to vaccine development, as protection against the two dominant clades is essential given the rapid mutation rate of HIV.

As a final backstop against mutations in the Gag p24 regions, additional CTL clones can be created against peptide sequences of the HIV polymerase gene pol. Pol codes for three enzymes: the RNA-Dependent DNA Polymerase, or Reverse Transcriptase, the Integrase, which inserts the proviral DNA genome into the host chromosome, and the Protease, which cleaves the Gag-Env precursor at the host cell membrane.

Enzymes are extremely sensitive to deleterious mutations, as they depend on strict secondary, tertiary, and quaternary structural interactions for their interactions with substrate and coenzyme factors. As expected, the HIV pol gene is the most conserved gene of all, with 88% exact sequence homology among diverse strains, compared with 20% in Env, and 66% in Gag p24 capsid protein. Although Pol products are not produced in the same quantities as structural proteins such as p17, p24, gp41, and gp120, enough peptide is captured by HLA MHC Class I proteins to be recognized by Pol-specific CTL. These CTL can provide an additional measure of protection against any mutant that could possibly avoid the eight Gag-specific CTL clones.

Since the mutation rate of HIV in estimated at 1 amino acid substitution per viral genome per replicative cycle, multiple backup CTL clones are required for an effective vaccine. Two concepts of Population Biology are at work in an HIV infection. The Red Queen Hypothesis states that for two organisms competing for the same resources, the organism with mutations that confer advantages (immune evasion), will prevail. The other side of the coin is Muller's Ratchet, which states that asexual organisms with a high recombinational rate will eventually develop populations that are less fit than the parental strains. Muller's Ratchet was demonstrated in HI infection by a group of researchers who performed multiple serial passages, and tested the resulting strains for infectivity. Nine out of ten virus progeny strains were less fit than the parental strain, only one became more infective. So, HIV is not the invincible enemy of popular thought. By using the described vaccine, we can force HIV into extinction by Muller's Ratchet. A strain would have to mutate in twelve separate regions of its most conserved proteins, and each mutation would have to yield infective virus. The probability is 1×10 raised to the 65^{th} power. There are an equal number of atoms in the known universe.

### Peptide Synthesis Procedure

Although many methods of synthesizing short chains of amino acids may be employed, the following method is described.

### Step 1 (Synthesis)

Reagent grade methylene chloride to be distilled from anhydrous potassium carbonate Reagent grade dimethylformamide to be treated with 0.4 nm molecular sieves 48 hours prior to use. Additional materials and sources: (All materials are Reagent-Grade unless otherwise specified).

| Material | Commercial Source |
|---|---|
| Isopropanol | Fisher Chemical |
| Triflouroacetic acid | Eastman Chemical |
| Dicyclohexylcarbodiimide | Vega Biochemicals |
| Hydroxybenzotriazole | Sigma Chemical |
| Protected amino acids | Vega |
| Benzhdyrlamine resin 0.33 mmol/g | Pierce Biochemical |

Side chains of threonine and glutamic acid to be protected with O-benzyl groups, tyrosine by ortho-bromobenzyl-oxycarbonyl, and cysteine with S-para-methoxybenzyl. The inital amino acid (e.g., aspartic acid), converted to butoxycarbonyl-B-benzylaspartic acid, and coupled to the bezhydralamine resin. Coupling reactions to be checked at each step using ninhydrin and/or picric acid at the end of the cycle. Double-coupling will be necessary residues of aspartic acid, cysteine, proline, tyrosine, and phenylalanine. An equimolar amount of butoxycarbonyl-asparaginine to be included to prevent formation of cyanoalanine.

Following the final amino acid coupling, the N-terminal butoxycarbonyl group to be removed and the resin vacuum dried overnight to yield the peptide resin (95-97% pure). The peptide can be cleaved from the resin and the side-chain protective groups removed by the treatment of 1.0 g of resin plus 1.0 ml anisole with 20 ml liquid HF for 1.0 hour at 4 C. After removal of HF at 4 C. with a stream of Nitrogen, the excess anisole can be removed by extraction with anhydrous ether (100) ml, with the resulting mixture to be exposed to high vacuum in the presence of NaOH pellets to remove any volatile HF. The crude peptide resin can be extracted with Nitrogen-deareated 5% HOAc (100 ml), to remove any remaining reagents. The resulting mixture to be diluted with water to 20% HOAc and put through a Sephadex G-10 column equilibrated with 20% HOAc. Final product to be lyophilized to yield final peptide

### Isolation of Mononuclear Cells and Monocytes

A. Materials

1. Leukocyte-enriched leukapheresis packs

2. Ficoll-Paque solution

3. RPMI-10 (Complete RPMI-1640 containing 10% heat-inactivated fetal bovine serum

4. RPMI-20 (Complete RPMI-1640 containing 20% heat-inactivated fetal bovine serum

5. Hanks Balanced Salt Solution (HBSS), Ca++ and Mg++ free

6. Conical centrifuge tubes

7. Tissue culture flasks

8. Fetal Bovine Serum (FBS), with or without heat inactivation (1 hr, 56 degrees C.)

9. Beckman GPR centrifuge with GH-3.7 horizontal rotor (or equivalent temperature-controlled centrifuge)

10. Additional reagents and equipment for counting cells and trypan blue exclusion for viability assay

B. Isolation of Mononuclear Cells

1. Dilute blood from the leukapheresis donor with HBSS (1:4 blood/HBSS).

2. Slowly layer 30 ml of the blood/HBSS mixture over 10 ml of the Ficoll-Paque solution in a 50-ml centrifuge tube. To maintain the Ficoll-Paque/blood interface, it is helpful to hold the centrifuge tube at a 45 degree angle.

3. Centrifuge 30 min. in a GH-3.7 rotor at 2000 rpm (900×g), 18 degrees C., with no brake.

4. Using a sterile pipet, remove the upper layer that contains the plasma and most of the platelets (FIG. 1). Using another pipet, transfer the mononuclear layer to another centrifuge tube. Wash cells by adding excess HBSS (3 times the volume of the mononuclear cell layer) to another centrifuge tube and centrifuging 10 minutes at 1300 rpm (400×g), at 18 to 20 degrees C. Remove supernatant, resuspend cells in HBSS, and repeat the wash once to remove most of the platelets

5. Resuspend mononuclear cells in complete RPMI-10. Count cells and determine viability by trypan blue exclusion.

C. Isolation of Monocytes by Adherence

Approximately 40% of the isolated mononuclear cells obtained in the previous protocol are monocytes and macrophages. Monocytes can be depleted form the isolated mononuclear cells suspension by taking advantage of the fact that monocytes adhere to plastic whereas lymphocytes do not.

1. Centrifuge mononuclear cells for 10 min. at 1400 rpm (300×g), at 18-20 degrees C. Remove supernatant and resuspend cell pellet in complete RPMI-20 to a final concentration of 2×10 6 cells/ml. Transfer 40 ml cells suspension to a 150 cm tissue culture flask.

2. Incubate horizontally for 1 hr. in a 37 degree C., 5% CO₂ humidified incubator.

3. Decant nonadherent lymphocytes into a centrifuge tube. Rinse tissue culture flask gently with 37 C. complete RPMI-10; add this wash to the centrifuge tube. Repeat steps 1 to 3 one more time.

4. Use monocytes to generate dendritic cells (see protocol “Generation of human dendritic cells from monocytes”).

C. Background and Critical Parameters

1. Peripheral blood is the primary source of lymphoid cells for the investigations of the human immune system. Its use is facilitated by Ficoll-Paque density-gradient centrifugation-a simple and rapid method of purifying peripheral blood mononuclear cells (PBMC) that takes advantage of the density differences between mononuclear cells and other elements found in the blood sample. Mononuclear cells and platelets collect on top of the Ficoll-Paque layer because they have a lower density. In contrast, erythrocytes and granulocytes have a higher density than Ficoll-Paque and collect at the bottom of the Ficoll-Paque layer. Platelets are separated from the mononuclear cells by subsequent washing or by centrifugation through a fetal-bovine serum cushion gradient that allows penetration of mononuclear cells but not platelets.

2. The yield and the degree of purity of the mononuclear cells depend on the percentage of contaminating granulocytes and platelets and the efficiency of erythrocyte removal. For maximum yield and purity, it is essential to remove all the material at the Ficoll-Paque interface and to ensure that no Ficoll-Paque solution or supernatant is removed with the sample. Including Ficoll-Paque will increase the granulocyte contamination, including supernatant will increase the platelet contamination.

3. When erythrocytes in whole blood are aggregated, some mononuclear cells are trapped in the clumps and, therefore, sediment with the erythrocytes. Diluting the blood 1:4 with HBSS reduces this tendency. Dilution gives a better monocyte yield and reduces the size of the red cell clumps.

4. Aggregation of erythrocytes is enhanced at higher temperatures (37 C.) which decreases yield, but at lower temperatures (4 C.) the rate of aggregation is decreased, increasing the time of separation. A temperature of 18 C. gives optimum results.

5. If problems are encountered when removing platelets form the lymphocyte fraction by the washing procedure, a second centrifugation in a 4-20% sucrose gradient layered over Ficoll-Paque will effectively remove the platelet contamination. The platelets will remain at the top of the sucrose gradient, and the lymphocytes will sediment through the sucrose gradient to the top of the Ficoll-Paque layer.

### Generation of Human Dendritic Cells from Monocytes

A. Materials

1. Leukocyte-enriched leukaphereisis packs

2. RPMI-10 (Complete RPMI-1640 containing 10% heat-inactivated fetal bovine serum)

3. 0.2 mM EDTA in PBS, Ca++ and Mg++ free

4. Recombinant human IL-4 (see Reagents and Solutions)

5. Inverted phase-contrast microscope

6. Sorvall centrifuge with H1000B rotor or equivalent

7. Conical centrifuge tubes

8. Recombinant human granulocyte-macrophage colony stimulation factor (GM-CSF) (see Reagents and Solutions)

9. Tissue Culture flasks

10. Additional reagents and equipment for isolation of peripheral blood monocytes followed by adherence to plastic (see protocol “Isolation of Mononuclear Cells”)

B Generation of Dendritic Cells

1. Isolate PBM from a leukaphereis preparation using Ficoll-Paque Plus (Pharmacia BioTech) density gradient centrifugation and plastic adherence (see protocol “Isolation of Mononuclear cells and Monocytes”.

2. Gently aspirate the medium that contains the nonadherent cells from the flasks and Wash each flask very gently with warm RPMI-10 to remove the nonadherent cells. Examine each flask using a phase-contrast microscope to determine the level of Contamination with small round lymphocytes.

3. Culture the adherent cells in complete RPMI-10 media containing 500 U/ml GM-CSF and 500 U/ml IL-4.

4. Add fresh complete RPMI-10 and cytokines to the cultures on day 3. Exchange about 75% of spent medium for fresh medium and cytokines.

5. On day 5 of incubation, add fresh complete RPMI-10 containing 500 U/ml GM-CSF and 500 U/Ml IL-4.

6. On day 7 of incubation, resuspend the cells by vigorous pipetting to break up the cellular aggregates and to wash the semi-adherent cells from the culture flasks.

7. Determine the purity of dendritic cells by immunofluoresence staining with flow cytometry analysis for MHC Class II, CD83,CD3, and CD14.

8. Cyropreserve half of the dendritic cells for a subsequent vaccination 2 weeks later.

9. Incubate the second aliquot of the dendritic cells for 2 hours in the presence of 50 ug/ml of peptide at room temperature. Incubate the dendritic cells with the 3 peptides separately.

10. Wash the peptide-loaded dendritic cells in sterile PBS and resuspend in injection-grade saline.

B. Reagents and Solutions

1. Recombinant human GM-CSF, 100×. Dilute (by <1:5) a 1 ug/ml vial of Human GM-CSF (Genzyme) in complete RPMI-10 to generate a solution Containing 50,000 U/ml GM-CSF. Divide into appropriately-sized aliquots and store at −70 degrees C. Use or store the solution immediately after reconstitution and dilution.

C Background and Critical Parameters

1. Dendritic Cells (DC) are highly-specialized antigen-presenting cells, which may be Isolated from human blood mononuclear cells. Although DC are widely dispersed throughout the body, they exhibit many common features: an irregular shape with extensive and elongated dendritic processes, a distinct cell-surface phenotype (including very high levels of MHC Class II antigens), low buoyant density, active motility, and the ability to stimulate the vigorous proliferation of unprimed T cells.

2. Isolating adequate numbers of pure DC from humans has always proven difficult because of their low frequency in blood (0.2%) and tissues. Mononuclear preparations enriched for DC are commonly isolated by negative selection, because DC in the blood lack surface antigens found on T cells, B cells, monocytes, and NK cells. The numbers of DC in cell preparations have been quantified primarily by determining the frequency of cells with a DC morphology, the frequency of cells with a limited phenotypic profile, or the ability to stimulate in mixed leukocyte reactions. However, the recent identification of CD83 as a marker preferentially expressed by human DC has greatly helped their isolation, identification, and characterization. CD83 is predominantly expressed on the surface of DC, including blood DC, skin Langerhans cells, and interdigitating reticulum cells present in the T cell zones of lymphoid organs CD83+ cells express the highest levels of MHC Class II antigens Compared with other leuckocyte lineages and are the most potent stimulator cells I allogenic mixed leukocyte reactions. It is therefore possible to define mature DC as being CD83+ cells, rather than relying principally on the cells having a DC-like morphology or having the greatest potency in a mixed leukocyte reaction. p0 3. Although mature blood DC normally represent about 0.2% of human blood mononuclear cells, their frequency can be greatly increased by the use of cell enrichment methods. More highly purified DC preparations can be obtained from these populations by sorting of fluorescence-labeled cells. Alternatively, DC can be generated from monocytes by culture with the appropriate cytokines. We chose the second method for several important reasons. Blood monocyte differentiation into a fairly homogenous population of functionally mature CD83+ DC under the influence of a specific cascade of cytokines can be divided into several stages. In the first stage, CD14+CD1a+ cells with a DC morphology are induced by GM-CSF/IL-4. In the second stage, the cells are induced by TNF-alpha to differentiate into CD83+C14-CD4+ cells. Finally, in a third stage, involving continued culture, the onocyte-derived CD83+ cells develop into cells with decreased CD1a expression that have a dermal dendritic cell phenotype. Depletion of CD14+ cells from mononuclear cell preparations depletes most DC precursors. The ability to generate DC from monocytes provides a new and simple method for generating functionally mature DC from a population of cells that are relatively easy to isolate to homogeneity, as opposed to CD34+ stem cells or other precursor cells.

4. The single most important factor in isolating adequate numbers of DC form human blood mononuclear cells is the availability of large numbers of starting cells. Small volumes of blood to not work well for the isolation of DC. Therefore, we plan to generate DC from leukapheresis-derived monocytes.

5. The technique-sensitive steps are those involving cell manipulation, because these can lead to large cell loses. The single most critical step is washing the tissue culture flasks free of non-adherent cells, pipetting with sufficient force to dislodge the non-adherent cells, while at the same time avoiding excessive force which can strip off adherence cells. It is essential to use warm medium in these steps, because cold medium detaches the adherent cells from the plastic.

6. The relative number of DC can be roughly assessed by phase-contrast microscopy of the cells in a hemacytometer. DC are large and have irregular, long membrane processes, while lymphocytes are small and round.

7. Isolation of DC from GM-CSF/IL-4-induced monocytes is less time-consuming than other methods. This procedure does not require a 7-day period of cell in addition to the time spent preparing the cells, and it is a more expensive approach, because of the cost of the recombinant cytokines. Nonetheless, the generation of human DC from monocytes can yield significant numbers of cells and a fairly homogenous population of DC, which has considerable advantages for clinical applications.

### Preparation of Attenuated Chikungunya Virus Culture for T-cell Proliferation

Chikungunya Virus (available at Walter Reed Army Hospital, Washington D.C.), is Passaged in African Green Monkey Kidney cells (AGMK), up to 5 times, with the final passage to be performed at 39.5 C. The resulting heat-attenuated viruses are inoculated on DBS-FRhl-2 roller flasks at a minimum of infection or MOI of 5×10−4. After adsorption for 1.5 hours at 35 C., the inoculum is removed and flasks are washed three times in Hank's Balanced Salt Solution (HBSS). Maintenance medium (200 ml/roller) consisting of Eagle's minimal essential medium with 0.25% human serum albumin, 0.22% NaHCO3, streptomycin (50 ug/ml), and neomycin (100 ug/ml). Medium on all flasks changed by day 4,and supernatant fluids harvested on day 6. Before centrifugation at 1,050×g for 20 minutes, human serum albumin to be added in a final concentration of 2.75%. Albumin pH to be adjusted to 7.6 before addition to viral fluids. As a final step in clarification, fluid to be filtered through a 0.45 um membrane filter. Samples then to be tested for adventitious microbes as described in Public Health Service regulations for live-attenuated viral vaccines (Code of Federal Regulations, Chapter 21, subchapter F, Biologics).

After removal of samples for testing and plaque assay, remaining volume to be held in ice baths in a 4 C refrigerator for 7 days pending results of safety testing and plaque assays. A final pool of virus to be made from the fluids of flasks containing less than 5 PFU/ml at 37 C., with no detectable large-plaque virus present. Average titer for vials to be 8.5×10 4 PFU/ml, then freeze-dried for use after neurovirulence testing. Intraspinal and intracerebral bihemispheric inoculation of 0.5 ml virus fluid into male rhesus monkeys with 2 controls receiving virus-free culture fluids. Monkeys to be observed daily for evidence of CNS involvement or other physical abnormalities. Following sacrifice, histological examination of lumbar and cervical cord, lower and upper medulla oblongata, mesencehpalon, and motor cortex to be made for viral pathology. If all results are negative, virus is ready to be used as T-cell activator.

### Administration of Dendritic Cells and Chikungunya Virus

Human subject to be infused by continues Intravenous catheter with 2×10 6 peptide-loaded DC. The following day, injection of 5 ml of virus culture fluid to be administered by hypodermic syringe. The subject will experience mild flu-like symptoms for 2-3 days as the cytokine cascade and subsequent T-cell activation takes place. After complete recovery, the subject will have 2 billion CTL specific for the conserved peptides. Any subsequent exposure to HIV containing these core peptides will provoke a memory CTL response, eradicating infected cells.

I claim:

1. A method of inducing a specific protective immune response to the Human Immunodeficiency Virus in a human being, comprising the steps of:
isolating antigen-presenting dendritic cells from said human being, and pulsing said dendritic cells with synthetic peptides that bind said human being's Class I Major Histocompatiblity types, said peptides corresponding to conserved structural and functional gene products of said Human Immunodeficiency Virus, and injecting said pulsed dendritic cells intravenously into said human being, and injecting said human being with substantially pure Chikungunya Virus.

2. The method of claim 1 wherein said substantially pure Chikungunya Virus is purified by passing said virus through living cells with an active transmembrane gradient; and passing said virus through subhuman primates to confirm the elimination of neurovirulence from the virus.

3. The method of claim 2 wherein said cells with an active transmembrane gradient are African Green Monkey Kidney Cells are infected with virus at less than 5 plaque-forming units/ml.
